(19) United States
(12) Patent Application Publication
(10) Pub. No.: US 2024/0407994 A1
(43) Pub. Date: Dec. 12, 2024

(54) COSMETIC COMPOSITIONS FOR USE IN SKIN CARE

(71) Applicant: GIGI COSMETIC LABORATORIES LTD, Petah Tikva (IL)

(72) Inventor: Haim ZER, Petah Tikva (IL)

(73) Assignee: GIGI COSMETIC LABORATORIES LTD, Petah Tikva (IL)

(21) Appl. No.: 18/702,343

(22) PCT Filed: Oct. 30, 2022
(86) PCT No.: PCT/IL2022/051150

§ 371 Date: Apr. 18, 2024

Related U.S. Application Data
(60) Provisional application No. 63/273,947, filed on Oct. 31, 2021.

(51) Int. Cl. A61K 8/64; A61K 8/73; A61K 8/9789; A61K 8/99; A61Q 17/04; A61Q 19/00; A61Q 19/08

(52) U.S. Cl. CPC A61K 8/64; A61K 8/735; A61K 8/9789; A61K 8/99; A61Q 17/04; A61Q 19/00; A61Q 19/007; A61Q 19/08

(57) ABSTRACT

Described herein are cosmetic compositions for the use in skin care that include superfoods, peptides, hyaluronic acids or derivative thereof, and prebiotics or postbiotics. The compositions described treat wrinkles of the skin, dryness of the skin, dark spots of the skin, improve firmness of the skin, improve elasticity of the skin, and decrease fading of the skin.

### FIELD

Provided herein are cosmetic compositions for the use in skin care.

### BACKGROUND

Skin care is care of the condition and appearance of the skin. Routine skin care may prevent skin conditions such as skin wrinkles, blemishes, roughness, dryness, fading, and dark circles. In addition, it may aid in improving the firmness, elasticity, and skin thickness.

Human skin is comprised of three main layers: the subcutaneous tissue, dermis and epidermis. The subcutaneous tissue is composed of fat, connective tissue, and large blood vessels. The dermis contains nerve endings, oil and sweat glands, and hair follicles. The epidermis is the outer layer of the skin which contains cells that produce pigment and protect the body from the penetration of foreign matter.

Superfoods are commonly referred to foods that contain compounds that may be beneficial to one's health. They may include vitamins, amino acids, minerals, antioxidants, and/or enzymes.

Hyaluronic acid (HA) is a hydrophilic macromolecule and acts as a “molecule sponge” due to its hygroscopic properties. HA is naturally found in the human body.

Described herein are unique topical compositions with the ability to penetrate the skin and rejuvenate it from within.

### SUMMARY

Described herein are compositions comprising a composition for treatment of the skin, comprising at least two components of a superfood group, a peptide group, a hyaluronic acid (HA) or derivative thereof, and a prebiotics & postbiotics group.

Additionally described herein are methods for treatment of skin conditions that includes, applying to the skin at least two components selected from: a superfood, a peptide, a hyaluronic acid or derivative thereof, and a prebiotics & postbiotics, thereby treating the skin condition.

### DETAILED DESCRIPTION

Unless otherwise noted, technical terms are used according to conventional usage. Definitions of common terms in pharmaceutical sciences can be found in Troy et al. Remington: The Science and Practice of Pharmacy. Published by Lippincott Williams & Wilkins, 2006. In case of conflict, the present specification, including explanations of terms, will control. In addition, all the materials, methods, and examples are illustrative and not intended to be limiting.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia and is used herein to indicate a non-limiting example. Thus, the abbreviation “e.g.” is synonymous with the term “for example.”

### Definitions

Combination: A treatment modality combining two or more treatments (therapies or agents). Combination therapy may involve administration of the two or more treatments at the same time, sequentially, or with a gap of time between the administrations. In combination therapy, although not always administered simultaneously, the biological effects of both of the drugs or treatments occur on the subject at relatively the same time. Combination therapy may involve two (or more) drugs or treatments in one dosage form or multiple drugs or treatments in separate dosage forms. Combination therapy may involve combining both agents in a single dosage form before administration, or administering the agents in separate dosage forms.

Postbiotic: an agent released from probiotic bacteria. Probiotics are beneficial microorganisms in human skin that keeps us healthy by improving metabolism, supporting immune function and controlling inflammation.

Prebiotic: an agent which serves as a food to increase growth and functional activity of a probiotic. Dietary fiber is an example of a prebiotic.

Prebiotic/Postbiotic complex: a composition comprising a prebiotic and a postbiotic agent. Occasionally, the composition may comprise a bacteria known to be a probiotic. An example of such a complex is Alpha-glucan oligosaccharide & Polymnia sonchifolia root juice & Maltodextrin & Lactobacillus.

Probiotic: bacteria which typically live in a human gut and can be helpful to digestion. Exemplary probiotics include Lactobacillus acidophilus. Probiotics are beneficial microorganisms in human skin that may improve metabolism, support immune function and control inflammation.

Superfood: Food additives which may include vitamins, minerals, antioxidants, amino acids and/or enzymes. Non-limiting examples of superfoods are: Morinda citrifolia Callus Culture Lysate, Punica Granatum Seed Cell Culture Lysate, Curcuma longa Callus Lysate, and Olea europaea Callus Culture Lysate.

Synergy: refers to a clinical observation wherein a combination of two or more components such as the described composition comprising a superfood, a peptide, a hyaluronic acid or derivative thereof, and a pre/postbiotic, when administered in combination, provides more than an additive effect of the administration of each component alone for example in the use of skin care.

Therapeutically effective amount: an amount of a compound sufficient to show benefit to the individual. This amount prevents, alleviates, abates, or otherwise reduces the severity of a symptom associated with a condition in a patient.

### DESCRIPTION OF EMBODIMENTS

According to an embodiment, the described night cream may optionally include at least two of the following components superfood mix: Morinda citrifolia callus culture lysate, punica granatum seed cell culture lysate, Curcuma longa callus lysate, citric acid, Olea europaea callus culture lysate; peptides: tetradecyl aminobutyroylvalylaminobutyric urea trifluoroacetate, palmitoyl tripeptide-5, palmitoyl dipeptide-5 diaminobutyroyl hydroxythreonine; HA: hydrolyzed hyaluronic acid 5-20 kda, 20-50 kda, 100-300 kda; 800-1,500 kda, 5,000-20,000 kda and niacinamide, succinic acid, and/or ascorbyl tetraisopalmitate.

According to an embodiment, the described night cream may alternatively include two, or more of the following components superfood mix: Morinda citrifolia callus culture lysate, Punica granatum seed cell culture lysate, Curcuma longa callus lysate, Olea europaea callus culture lysate; peptides: palmitoyl tripeptide-7, palmitoyl tetrapeptide-1, acetyl hexapeptide-8); HA: hydrolyzed hyaluronic acid 5-20 kda, 20-50 kda, 100-300 kda, 800-1500 kda); probiotic: lactobacillus; and may further include niacinamide, and/or ascorbyl tetraisopalmitate.

According to an embodiment, the described day cream composition may optionally include at least one or more of the following components super food mix: Morinda citrifolia callus culture, Punica granatum seed cell culture lysate, Curcuma longa callus lysate, Olea europaea callus culture lysate; peptides: tetradecyl aminobutyroylvalylaminobutyric urea trifluoroacetate, palmitoyl tripeptide-5, palmitoyl dipeptide-5 diaminobutyroyl hydroxythreonine, dipeptide diaminobutyroyl benzylamide diacetate, acetyl hexapeptide-8; HA: hydrolyzed hyaluronic acid 5-20 kda, 20-50 kda, 100-300 kda), and may further include niacinamide, succinic acid, and/or ascorbyl tetraisopalmitate.

According to an embodiment, the described day cream composition may alternatively optionally include one or more of the following components superfood mix: Morinda citrifolia callus culture lysate, Punica granatum seed cell culture lysate, Curcuma longa callus lysate, Olea europaea callus culture lysate; peptides: palmitoyl tripeptide-7, palmitoyl tetrapeptide-1, acetyl hexapeptide-8; HA: hydrolyzed hyaluronic acid 5-20 kda, 20-50 kda, 100-300 kda, 800-1500 kda; probiotic: lactobacillus; and may further comprise niacinamide, and/or ascorbyl tetraisopalmitate.

According to an embodiment, the described softening skin cleanser composition may optionally include at least one of the following components super food mix: Morinda citrifolia callus culture lysate, Punica granatum seed cell culture lysate, Curcuma longa callus lysate, Olea europaea callus culture lysate; and/or maltodextrin, lecithin, Alpinia officinarum root extract.

According to an embodiment, the described softening skin cleanser composition may further include at least one of the following components superfood mix: Morinda citrifolia callus culture lysate, Punica granatum seed cell culture lysate, Curcuma longa callus lysate, Olea europaea callus culture lysate; peptides: palmitoyl tripeptide-7, palmitoyl tetrapeptide-1, acetyl hexapeptide-8): HA: hydrolyzed hyaluronic acid 5-20 kDa, 20-50 kDa, 100-300 kDa, 800-1500 kDa; and probiotic: Lactobacillus.

According to an embodiment, the described neck cream composition may optionally include the following components super food mix: Morinda citrifolia Callus Culture Lysate, Punica Granatum Seed Cell Culture Lysate, Curcuma longa Callus, Olea europaea Callus Culture Lysate; peptide: Tetradecyl Aminobutyroylvalylaminobutyric Urea Trifluoroacetate, Palmitoyl Tripeptide-5, Palmitoyl Dipeptide-5 Diaminobutyroyl Hydroxythreonine; HA: Hydrolyzed hyaluronic acid 5-20 kDa, 20-50 kDa, 100-300 kDa; and may further include Niacinamide, Succinic acid, Palmitoyl Glycine, Macrocystis Pyrifera Extract, PVP, Hydrolyzed Vegetable Protein, Coco-Glucoside, 1,2-Octanediol, Alcohol, and/or Glaucine).

According to an embodiment, the described neck cream composition may alternatively include at least one of the following components superfood mix: Morinda citrifolia Callus Culture Lysate, Citric Acid, Punica Granatum Seed Cell Culture Lysate Curcuma longa Callus Lysate, Olea Europaea Callus Culture Lysate; peptides: Palmitoyl Tripeptide-7, Palmitoyl Tetrapeptide-1, Acetyl Hexapeptide-8; HA: Hydrolyzed hyaluronic acid 5-20 kDa, 20-50 kDa, 100-300 kDa, 800-1500 kDa; probiotic: Lactobacillus; and may further comprise Niacinamide, Ascorbyl tetraisopalmitate, Palmitoyl Glycine, Macrocystis Pyrifera Extract, PVP, Hydrolyzed Vegetable Protein, Coco-Glucoside, 1,2-Octanediol, Alcohol, and/or Glaucine.

According to an embodiment, the described skin serum may be a skin firming skin booster serum that may include at least one of the following components super food mix: Morinda citrifolia Callus Culture Lysate, Punica Granatum Seed Cell Culture Lysate, Curcuma Longa Callus Lysate, Europaea Callus Culture Lysate; peptide: Tetrapeptide-4; HA: Hydrolyzed hyaluronic acid 5-20 kDa, 20-50 kDa, 100-300 kDa, and may further include Macrocystis Pyrifera Extract, PVP, and/or Hydrolyzed Vegetable Protein.

According to an embodiment, the described skin serum may alternatively include at least one of the following components super food mix: Morinda citrifolia Callus Culture Lysate, Punica Granatum Seed Cell Culture Lysate, Curcuma longa Callus Lysate, Olea Europaea Callus Culture Lysate; peptides: Palmitoyl Tripeptide-7, Palmitoyl Tetrapeptide-1. Acetyl Hexapeptide-8; HA: Hydrolyzed hyaluronic acid 5-20 kDa, 20-50 kDa, 100-300 kDa, 800-1500 kDa; and probiotic: Lactobacillus.

According to an embodiment, the described skin serum may be a skin brightener serum. The skin brightener serum may be a water-based formulation, that may include at least of the following components super food mix: Morinda citrifolia Callus Culture Lysate, Punica Granatum Seed Cell Culture Lysate, Curcuma longa Callus Lysate, Olea Europaea Callus Culture Lysate; and may further include Alpha-Arbutin, Ethyl Ascorbic Acid, Niacinamide, tranexamic acid, and/or Pisum Sativum Extract. The skin brightener serum may be an oil-based formulation, that may partially include some of the above components, and may further include at least one of the following components Dimethyl Isosorbide, Hydroxypinacolone Retinoate, Ascorbyl tetraisopalmitate, and/or Daucus Carota Seed Oil.

According to an embodiment, the described vitamin-C serum may include at least one of the following components super food mix: Morinda citrifolia Callus Culture Lysate, Punica Granatum Seed Cell Culture Lysate, Curcuma longa Callus Lysate, Olea Europaea Callus Culture Lysate; peptides: Tetradecyl Aminobutyroylvalylaminobutyric Urea Trifluoroacetate, Palmitoyl Tripeptide-5, Palmitoyl Dipeptide-5 Diaminobutyroyl Hydroxythreonine; HA: Hydrolyzed hyaluronic acid 5-20 kDa, 20-50 kDa, 100-300 kDa), and/or Ethyl Ascorbic Acid.

According to an embodiment, the described vitamin-C serum may alternatively include at least one of the following components super food mix: Morinda citrifolia Callus Culture Lysate, Punica Granatum Seed Cell Culture Lysate, Curcuma longa Callus Lysate, Olea Europaea Callus Culture Lysate; peptides: Palmitoyl Tripeptide-7, Palmitoyl Tetrapeptide-1, Acetyl Hexapeptide-8; HA: Hydrolyzed hyaluronic acid 5-20 kDa, 20-50 kDa, 100-300 kDa, 800-1500 kDa; probiotic: Lactobacillus; and/or Ethyl Ascorbic Acid.

According to an embodiment, the described probiotic soothing anti-aging eye cream may include at least one of the following components super food Morinda citrifolia Callus Culture Lysate, Punica Granatum Seed Cell Culture Lysate, Curcuma longa Callus Lysate, Glycerin, Olea Europaea Callus Culture Lysate; peptide: Palmitoyl Tripeptide-5; HA: Hydrolyzed hyaluronic acid 5-20 kDa, 20-50 kDa, 100-300 kDa; and may further include Niacinamide, Succinic acid, Panthenol, Sodium Hyaluronate, Dunaliella Salina Extract, Phenoxyethanol, Sodium Benzoate, Ethylhexylglycerin, Potassium Sorbate, Pantolactone, Albizia julibrissin Bark Extract, Darutoside and/or, (Palmitoyl Glycine).

According to an embodiment, the described probiotic soothing anti-aging eye cream may alternatively include at least one of the following components super food Morinda Citrifolia Callus Culture Lysate, Punica Granatum Seed Cell Culture Lysate, Curcuma longa Callus Lysate Olea Europaea Callus Culture Lysate; peptide: Palmitoyl Tripeptide-7, Palmitoyl Tetrapeptide-1, Acetyl Hexapeptide-8; HA: Hydrolyzed hyaluronic acid 5-20 kDa, 20-50 kDa, 100-300 kDa, 800-1500 kDa; probiotic: Lactobacillus; and may further include Niacinamide and/or Ethyl Ascorbic Acid.

According to an embodiment, the described cosmetic facemask may include at least one of the following components super food Morinda citrifolia Callus Culture Lysate, Punica Granatum Seed Cell Culture Lysate, Curcuma longa Callus Lysate, Glycerin, Olea Europaea Callus Culture Lysate; peptide: Tetradecyl Aminobutyroylvalylaminobutyric Urea Trifluoroacetate, Palmitoyl Tripeptide-5, Palmitoyl Dipeptide-5 Diaminobutyroyl Hydroxythreonine; and HA: Hydrolyzed hyaluronic acid 5-20 kDa, 20-50 kDa, 100-300 kDa.

According to an embodiment, the described cosmetic facemask may alternatively include at least one of the following components super food Morinda citrifolia Callus Culture Lysate, Punica Granatum Seed Cell Culture Lysate, Curcuma longa Callus Lysate, Olea Europaea Callus Culture Lysate; peptide: Palmitoyl Tripeptide-7, Palmitoyl Tetrapeptide-1, Acetyl Hexapeptide-8; HA: Hydrolyzed hyaluronic acid 5-20 kDa, 20-50 kDa, 100-300 kDa, 800-1500 kDa; probiotic: Lactobacillus and may further include 5% mandelic acid, 5% lactic acid, and/or 2% salicylic acid.

According to an embodiment, the described composition includes at least two of the following components superfood, peptide, HA, and probiotic. The composition administered in an amount as to obtain a synergistic effect. A synergistic effect refers to a clinical observation wherein a combination of the components of the composition, when administered in combination, provides more than an additive effect of administration of each component alone. The synergy may be seen in treatment of skin dryness, wrinkles, blemishes, fading, and dark circles, and may been seen to synergistically improve firming, elasticity, thickness of the skin, and reduce roughness of the skin.

The following examples are provided to illustrate certain particular features and/or embodiments. These examples should not be construed to limit the disclosure to the particular features or embodiments described.

### EXAMPLES

### Example 1: Night Cream Composition

The following example describes a composition for topical application to be used at night. The components of the night composition are shown in Table 1 below.

TABLE 1
Components of Beauty Sleep Night Cream Composition

| INGREDIENTS | % |
|---|---|
| Water (Aqua)/ | 50.7013 |
| Glycerin | 6.4426 |
| Butyrospermum parkii (Shea) Butter | 6 |
| Cetyl Alcohol | 5 |
| Glyceryl Stearate Citrate | 3.5 |
| Isononyl Isononanoate | 3 |
| Propanediol | 3 |
| Cetearyl Alcohol | 2.8 |
| Cetearyl Ethylhexanoate | 2.5 |
| Maltodextrin | 1.624 |
| Coco-Caprylate | 1.4993 |
| Alpha-glucan oligosaccharide | 1.421 |
| Acetyl Hexapeptide-8 | 1.015 |
| Curcuma Longa (Turmeric) Callus Lysate | 1.015 |
| Olea Europaea (Olive) Callus Culture Lysate | 1.015 |
| Niacinamide | 1 |
| Sorbitan tristearate | 1 |
| Sodium hyaluronate | 0.9652 |
| Glyceryl Caprylate | 0.7 |
| Cetearyl Glucoside | 0.686 |
| Morinda Citrifolia Callus Culture Lysate | 0.609 |
| Punica Granatum Seed Cell Culture Lysate | 0.609 |
| Phenyl trimethicone | 0.6 |
| aminomethyl propanol | 0.475 |
| p-Anisic Acid | 0.4 |
| Butylene Glycol | 0.3857 |
| Polymnia sonchifolia root juice | 0.3857 |
| Carbomer/ | 0.31218 |
| Fragrance (Parfum) | 0.3 |
| Aloe Barbadensis Leaf Juice Powder | 0.2 |
| Ascorbyl Tetraisopalmitate | 0.2 |
| Citric Acid | 0.1116 |
| Linoleic Acid | 0.1038 |
| Plukenetia Volubilis Seed Oil | 0.1 |
| Oleic Acid | 0.096 |
| Tocopherol | 0.07392 |
| Phytic Acid | 0.05 |
| Helianthus Annuus (Sunflower) Seed Oil | 0.03 |
| Lactobacillus | 0.0203 |
| Sodium Lactate | 0.0203 |
| Glucose | 0.0105 |
| Coco-Glucoside | 0.01015 |
| Cyamopsis Tetragonoloba (Guar) Gum | 0.005075 |
| Xanthan Gum | 0.005075 |
| Linolenic Acid | 0.002 |
| Palmitoyl Tripeptide-1 | 0.000203 |
| Palmitoyl Tetrapeptide-7 | 0.0001015 |
| TOTAL | 100.0000 |

### Synthesis of Night Cream Composition

Heat the water until 75 C and add Carbomer via a mixer. Homogenize 15 minutes until it dissolves. Add the components: Niacinamide, Propanediol, Glycerin, Aloe Barbadensis Leaf Juice Powder, Phytic acid, consecutively while mixing. Separately mix the components: Water, p-Anisic Acid, Aminomethyl propanol, the pH must reach between 8-9 pH. and add it to the mixer. Separately heat the components: Cetearyl Ethylhexanoate, Isononyl Isononanoate, Phenyl trimethicone, Butyrospermum parkii (Shea) Butter, Glyceryl Stearate Citrate, Cetearyl Alcohol, Cetyl Alcohol, Coco-Caprylate, Sorbitan tristearate, Glyceryl Caprylate, Cetearyl Glucoside, Plukenetia volubilis Seed Oil until 75 C and add to the mixture by means of a mixer. Homogenize 20 minutes and start cooling while stirring. At 50 C add the components: Sodium hyaluronate 5-20 kDa, 20-50 kDa, 100-300 kDa, 800-1500 kDa by means of a mixer, and mix. At 40 C add the components: Ascorbyl Tetraisopalmitate, Linoleic Acid, Oleic Acid, Linolenic Acid, Tocopherol consecutively by means of a mixer. Separately mix the components: Water, Punica Granatum Seed Cell Culture Lysate, Polymnia sonchifolia root juice, Maltodextrin, Lactobacillus until it dissolves and add to the mixer at 35 C. Add sequentially Curcuma longa (Turmeric) Callus Lysate, Olea europaea (Olive) Callus Culture Lysate, Morinda citrifolia Callus Culture Lysate. Separately mix the components: Water, Acetyl Hexapeptide-8, Butylene Glycol, Coco-Glucoside, Palmitoyl Tripeptide-1, Palmitoyl Tetrapeptide-7 and add it to the mixer. check the pH in the mixer, which must reach between 6.5-7.5 pH. Add Parfum.

### Example 2: Day Cream Composition

The following example describes a composition for topical application to be used during the day. The components of the day cream composition are shown in Table 2 below.

TABLE 2
Components of Day Cream Composition

| INGREDIENTS | % |
|---|---|
| Water (Aqua) | 54.403167 |
| Glycerin | 7.442655 |
| Cetyl Alcohol | 4 |
| Glyceryl Stearate Citrate | 3.5 |
| Isononyl Isononanoate | 3 |
| Propanediol | 3 |
| Cetearyl Alcohol | 2.8 |
| Cetearyl Ethylhexanoate | 2.5 |
| Butyrospermum parkii (Shea) Butter | 2 |
| Maltodextrin | 1.624 |
| Sorbitan tristearate | 1.5 |
| Coco-Caprylate | 1.499325 |
| Alpha-glucan oligosaccharide | 1.421 |
| Acetyl Hexapeptide-8 | 1.015 |
| Curcuma Longa (Turmeric) Callus Lysate | 1.015 |
| Olea Europaea (Olive) Callus Culture Lysate | 1.015 |
| Niacinamide | 1 |
| Sodium hyaluronate | 0.9652 |
| Glyceryl Caprylate | 0.7 |
| Cetearyl Glucoside | 0.686 |
| Morinda Citrifolia Callus Culture Lysate | 0.609 |
| Punica Granatum Seed Cell Culture Lysate | 0.609 |
| Phenyl trimethicone | 0.6 |
| aminomethyl propanol | 0.475 |
| p-Anisic Acid | 0.4 |
| Butylene Glycol | 0.3857 |
| Polymnia sonchifolia root juice | 0.3857 |
| Carbomer | 0.31218 |
| Fragrance (Parfum) | 0.3 |
| Aloe Barbadensis Leaf Juice Powder | 0.2 |
| Ascorbyl Tetraisopalmitate | 0.2 |
| Citric Acid | 0.11165 |
| Plukenetia Volubilis Seed Oil | 0.1 |
| Tocopherol | 0.07372 |
| Phytic Acid | 0.05 |
| Helianthus Annuus (Sunflower) Seed Oil | 0.03 |
| Lactobacillus | 0.0203 |
| Sodium Lactate | 0.0203 |
| Glucose | 0.0105 |
| Coco-Glucoside | 0.01015 |
| Cyamopsis Tetragonoloba (Guar) Gum | 0.005075 |
| Xanthan Gum | 0.005075 |
| Palmitoyl Tripeptide-1 | 0.000203 |
| Palmitoyl Tetrapeptide-7 | 0.0001015 |
| TOTAL | 100.00000 |

### Synthesis of Day Cream Composition

Heat the water until 75 C and add Carbomer via a mixer. Homogenize 15 minutes until it dissolves. Add the components: Niacinamide, Propanediol, Glycerin, Aloe Barbadensis Leaf Juice Powder, Phytic acid, consecutively while mixing. Separately mix the components: Water, p-Anisic Acid, Aminomethyl propanol, the pH must reach between 8-9 pH. and add it to the mixer. Separately heat the components: Cetearyl Ethylhexanoate, Isononyl Isononanoate, Phenyl trimethicone, Butyrospermum parkii (Shea) Butter, Glyceryl Stearate Citrate, Cetearyl Alcohol, Cetyl Alcohol, Coco-Caprylate, Sorbitan tristearate, Glyceryl Caprylate, Cetearyl Glucoside, Plukenetia volubilis Seed Oil until 75 C and add to the mixture by means of a mixer. Homogenize 20 minutes and start cooling while stirring. At 50 C add the components: Sodium hyaluronate 5-20 kDa, 20-50 kDa, 100-300 kDa, 800-1500 kDa by means of a mixer, and mix. At 40 C add the components: Ascorbyl Tetraisopalmitate, Tocopherol consecutively by means of a mixer. Separately mix the components: Water, Punica Granatum Seed Cell Culture Lysate, Polymnia sonchifolia root juice, Maltodextrin, Lactobacillus until it dissolves and add to the mixer at 35 C. Add sequentially Curcuma longa (Turmeric) Callus Lysate, Olea europaea (Olive) Callus Culture Lysate, Morinda citrifolia Callus Culture Lysate. Separately mix the components: Water, Acetyl Hexapeptide-8, Butylene Glycol, Coco-Glucoside, Palmitoyl Tripeptide-1, Palmitoyl Tetrapeptide-7 and add it to the mixer. check the pH in the mixer, which must reach between 6.5-7.5 pH. Add Parfum.

### Example 3: Polishing Cleanser Composition

The following example describes a composition for topical application as a skin polishing cleanser. The components of the polishing cleanser composition are shown in Table 3 below.

TABLE 3
Components of the polishing cleanser

| INGREDIENTS | % |
|---|---|
| Water (Aqua) | 62.80967 |
| Sodium Coceth Sulfate | 7.2 |
| Glycerin | 5.442655 |
| Decyl Glucoside | 2.74 |
| Cetyl Palmitate | 2.5 |
| Acrylates/Palmeth-25 Acrylate Copolymer | 2.25 |
| Maltodextrin | 1.628255 |
| Hydrogenated Castor oil | 1.5 |
| Glyceryl Stearate | 1.425 |
| Alpha-glucan oligosaccharide | 1.421 |
| Acetyl Hexapeptide-8 | 1.015 |
| Curcuma Longa (Turmeric) Callus Lysate | 1.015 |
| Olea Europaea (Olive) Callus Culture Lysate | 1.015 |
| Sodium hyaluronate | 0.9652 |
| Morinda Citrifolia Callus Culture Lysate | 0.609 |
| Punica Granatum Seed Cell Culture Lysate | 0.609 |
| Lauryl Glucoside | 0.51 |
| Sodium Lauryl Glucose Carboxylate | 0.51 |
| Fragrance (Parfum) | 0.5 |
| Rubus idaeus (raspberry) seed powder | 0.5 |
| Vaccinium macrocarpon (cranberry) fruit powder | 0.5 |
| Ethylhexylglycerin | 0.4 |
| Butylene Glycol | 0.3857 |
| Polymnia sonchifolia root juice | 0.3857 |
| Pentylene Glycol | 0.35 |
| Caprylyl Glycol | 0.30003 |
| Propanediol | 0.3 |
| Aloe Barbadensis Leaf Juice Powder | 0.25 |
| Pinus Sylvestris Leaf Extract | 0.25 |
| Caprylhydroxamic Acid | 0.15 |
| Sodium Chloride | 0.12 |
| Citric Acid | 0.11165 |
| Cocos Nucifera (Coconut) Fruit Extract | 0.1 |
| Stearic Acid | 0.075 |
| C10 alcohol | 0.025 |
| Phytic Acid | 0.025 |
| Lactobacillus | 0.0203 |
| Sodium Lactate | 0.0203 |
| Sorbic Acid | 0.015 |
| Carbomer | 0.01218 |
| Coco-Glucoside | 0.01015 |
| Glucose | 0.01 |
| Cyamopsis Tetragonoloba (Guar) Gum | 0.005075 |
| Xanthan Gum | 0.005075 |
| Lecithin | 0.0035 |
| Tocopherol | 0.003045 |
| Alpinia Officinarum Root Extract | 0.0021 |
| Palmitoyl Tripeptide-1 | 0.000203 |
| Palmitoyl Tetrapeptide-7 | 0.0001015 |
| Xylitol | 0.000065 |
| Caprylic Acid | 0.00005 |
| TOTAL | 100.00000 |

### Synthesis of Polishing Cleanser Composition

Heat Water, Phytic Acid, Glycerin, Aloe Barbadensis Leaf Juice Powder until 75 C. Separately heat the components: Glyceryl Stearate, Cetyl Palmitate, Hydrogenated Castor oil until 75 C. Mix them via a mixer. Homogenize 15-20 minutes and start cooling while stirring. At 50 C add the components: Sodium hyaluronate 5-20 kDa, 20-50 kDa, 100-300 kDa, 800-1500 kDa by means of a mixer, and mix. Add sequentially Sodium Lauryl Glucose Carboxylate, Lauryl Glucoside, Sodium Coceth Sulfate, Decyl Glucoside. Separately, mix the components: Water, Acrylates/Palmeth-25 Acrylate Copolymer and add it to the mixer. Add sequentially the components: Propanediol, Pentylene Glycol, Cocos nucifera (Coconut) Fruit Extract, Pinus sylvestris Leaf Extract, Caprylyl Glycol, Caprylhydroxamic Acid, Ethylhexylglycerin. Separately mix the components: Water, Punica Granatum Seed Cell Culture Lysate, Polymnia sonchifolia root juice, Maltodextrin, Lactobacillus, Lecithin, Alpinia Officinarum Root Extract until it dissolves and add to the mixer at 35 C. Add sequentially Curcuma longa (Turmeric) Callus Lysate, Olea europaea (Olive) Callus Culture Lysate, Morinda citrifolia Callus Culture Lysate. Separately mix the components: Water, Acetyl Hexapeptide-8, Butylene Glycol, Coco-Glucoside, Palmitoyl Tripeptide-1, Palmitoyl Tetrapeptide-7 and add it to the mixer. Check the pH in the mixer, which must reach between 6.9-7.3 pH. Add Parfum. After 24 hours add the components: Rubus idaeus (raspberry) seed powder, Vaccinium macrocarpon (cranberry) fruit powder.

### Example 4: Neck Cream Composition

The following example describes a composition for topical application as a revitalizing neck cream. The components of the neck cream composition are shown in Table 4 below.

TABLE 4
Components of the Neck Cream

| INGREDIENTS | % |
|---|---|
| Water (Aqua) | 50.678307 |
| Glycerin | 8.792655 |
| Cetyl Alcohol | 4 |
| Glyceryl Stearate Citrate | 3.5 |
| Isononyl Isononanoate | 3 |
| Propanediol | 3 |
| Cetearyl Alcohol | 2.8 |
| Cetearyl Ethylhexanoate | 2.5 |
| Butyrospermum parkii (Shea) Butter | 2 |
| Maltodextrin | 1.624 |
| Sorbitan tristearate | 1.5 |
| Coco-Caprylate | 1.499325 |
| Alpha-glucan oligosaccharide | 1.421 |
| Acetyl Hexapeptide-8 | 1.015 |
| Curcuma Longa (Turmeric) Callus Lysate | 1.015 |
| Olea Europaea (Olive) Callus Culture Lysate | 1.015 |
| Niacinamide | 1 |
| Soybean (Glycine soja) Oil | 1 |
| Palmitoyl Glycine | 0.99 |
| Sodium hyaluronate | 0.9652 |
| Glyceryl Caprylate | 0.7 |
| Cetearyl Glucoside | 0.686 |
| Morinda Citrifolia Callus Culture Lysate | 0.609 |
| Punica Granatum Seed Cell Culture Lysate | 0.609 |
| PHENYL Trimethicone | 0.6 |
| aminomethyl propanol | 0.475 |
| p-Anisic Acid | 0.4 |
| Butylene Glycol | 0.3857 |
| Polymnia sonchifolia root juice | 0.3857 |
| Carbomer | 0.31218 |
| Fragrance (Parfum) | 0.3 |
| Aloe Barbadensis Leaf Juice Powder | 0.2 |
| Ascorbyl Tetraisopalmitate | 0.2 |
| Macrocystis Pyrifera (Kelp) Extract | 0.12 |
| Citric Acid | 0.11365 |
| Plukenetia Volubilis Seed Oil | 0.1 |
| Tocopherol | 0.07372 |
| Coco-Glucoside | 0.07015 |
| PVP | 0.06 |
| Phytic Acid | 0.05 |
| Phenoxyethanol | 0.042 |
| Caprylyl Glycol | 0.03 |
| Helianthus Annuus (Sunflower) Seed Oil | 0.03 |
| Hydrolyzed Vegetable Protein | 0.03 |
| Lactobacillus | 0.0203 |
| Sodium Lactate | 0.0203 |
| Alcohol | 0.02 |
| Sodium Citrate | 0.013 |
| Glucose | 0.0105 |
| Cyamopsis Tetragonoloba (Guar) Gum | 0.005075 |
| Xanthan Gum | 0.005075 |
| Potassium Sorbate | 0.0045 |
| Glaucine | 0.002 |
| Sodium Benzoate | 0.00105 |
| Ethylhexylglycerin | 0.00021 |
| Palmitoyl Tripeptide-1 | 0.000203 |
| Palmitoyl Tetrapeptide-7 | 0.0001015 |
| Biotin | 0.0001 |
| TOTAL | 100.00000 |

### Synthesis of Neck Cream Composition

Heat the water until 75 C and add Carbomer via a mixer. Homogenize 15 minutes until it dissolves. Add the components: Niacinamide, Propanediol, Glycerin, Aloe Barbadensis Leaf Juice Powder, Phytic acid, consecutively while mixing. Separately mix the components: Water, p-Anisic Acid, Aminomethyl propanol, the pH must reach between 8-9 pH. and add it to the mixer. Separately heat the components: Cetearyl Ethylhexanoate, Isononyl Isononanoate, Phenyl trimethicone, Butyrospermum parkii (Shea) Butter, Glyceryl Stearate Citrate, Cetearyl Alcohol, Cetyl Alcohol, Coco-Caprylate, Sorbitan tristearate, Glyceryl Caprylate, Cetearyl Glucoside, Soybean (Glycine soja) Oil, Plukenetia volubilis Seed Oil, Palmitoyl Glycine until 75 C and add to the mixture by means of a mixer. Homogenize 20 minutes and start cooling while stirring. At 50 C add the components: Sodium hyaluronate 5-20 kDa, 20-50 kDa, 100-300 kDa, 800-1500 kDa by means of a mixer, and mix. At 40 C add the components: Ascorbyl Tetraisopalmitate, Linoleic, Tocopherol consecutively by means of a mixer. Add the components: Coco-Glucoside, Coco-Glucoside, Macrocystis Pyrifera (Kelp) Extract, Hydrolyzed Vegetable Protein, PVP, Alcohol. Separately mix the components: Water, Punica Granatum Seed Cell Culture Lysate, Punica Granatum Seed Cell Culture Lysate, Polymnia sonchifolia root juice, Maltodextrin, Lactobacillus until it dissolves and add to the mixer at 35 C. Add sequentially Curcuma longa (Turmeric) Callus Lysate, Olea europaea (Olive) Callus Culture Lysate, Morinda citrifolia Callus Culture Lysate. Separately mix the components: Water, Acetyl Hexapeptide-8, Butylene Glycol, Coco-Glucoside, Palmitoyl Tripeptide-1, Palmitoyl Tetrapeptide-7 and add it to the mixer. check the pH in the mixer, which must reach between 6.5-7.5 pH. Add Parfum.

### Example 5: Skin Serum Composition

The following example describes a composition for topical application as a skin hydrating and firming serum. The components of the skin serum composition are shown in Table 5 below.

TABLE 5
Components of Skin Serum

| INGREDIENTS | % |
|---|---|
| Water (Aqua) | 53.835 |
| Lactic Acid | 8.85 |
| Propanediol | 5.3 |
| Isopentyldiol | 4.95 |
| aminomethyl propanol | 4.94 |
| Glycerin | 4.64265 |
| Pentylene Glycol | 3.35 |
| Ethoxydiglycol | 2.5 |
| Alpha-glucan oligosaccharide | 1.421 |
| Maltodextrin | 1.421 |
| Acetyl Hexapeptide-8 | 1.015 |
| Curcuma Longa (Turmeric) Callus Lysate | 1.015 |
| Olea Europaea (Olive) Callus Culture Lysate | 1.015 |
| 1,2-Hexanediol | 1 |
| Sodium hyaluronate | 0.9652 |
| Morinda Citrifolia Callus Culture Lysate | 0.609 |
| Punica Granatum Seed Cell Culture Lysate | 0.609 |
| Levulinic Acid | 0.4 |
| Butylene Glycol | 0.3857 |
| Polymnia sonchifolia root juice | 0.3857 |
| Fragrance (Parfum) | 0.3 |
| Pinus Sylvestris Leaf Extract | 0.25 |
| Maltodextrin | 0.203 |
| Sodium Levulinate | 0.2 |
| Caprylhydroxamic Acid | 0.15 |
| Citric Acid | 0.11165 |
| Cocos Nucifera (Coconut) Fruit Extract | 0.1 |
| Lactobacillus | 0.0203 |
| Sodium Lactate | 0.0203 |
| Carbomer | 0.01218 |
| Coco-Glucoside | 0.01015 |
| Cyamopsis Tetragonoloba (Guar) Gum | 0.005075 |
| Xanthan Gum | 0.005075 |
| Tocopherol | 0.003045 |
| Palmitoyl Tripeptide-1 | 0.000203 |
| Palmitoyl Tetrapeptide-7 | 0.0001015 |
| TOTAL | 100.00000 |

### Synthesis of Skin Serum

Mix the components: Water, Alpha-glucan oligosaccharide, Polymnia sonchifolia root juice, Maltodextrin, Lactobacillus, Sodium hyaluronate 5-20 kDa, 20-50 kDa, 100-300 kDa, 800-1500 kDa, Isopentyldiol, Propanediol, Ethoxydiglycol, Pentylene Glycol, 1,2-Hexanediol, Cocos nucifera (Coconut) Fruit Extract, Pinus sylvestris Leaf Extract until it dissolves. Add to the mixer Levulinic Acid, Glycerin, Sodium Levulinate. Add to the mixer Lactic acid and aminomethyl propanol in small parts, check the pH after each part, the pH must reach between 3.5-4.0. Separately, mix the components: Water, Curcuma longa (Turmeric) Callus Lysate, Olea europaea (Olive) Callus Culture Lysate, Morinda citrifolia Callus Culture Lysate, Punica Granatum Seed Cell Culture Lysate at room temperature until it dissolves and add to mixer. Separately, mix the components: Water, Acetyl Hexapeptide-8, Butylene Glycol, Coco-Glucoside, Palmitoyl Tripeptide-1, Palmitoyl Tetrapeptide-7 until it dissolves and add it to the mixer. Check pH, which must reach 3.8-4.5 pH.

### Example 6: Safety of Compositions

The compositions disclosed above were tested in the following tests: Single Insult Patch Test, by applying the product to healthy adult skin (arm) of 50 volunteers for 48 hours. Skin was evaluated 1, 24 and 48 hours after application.

For each of the 5 compositions described in the examples 1-5 above, no negative observed reaction was observed, and no volunteers dropped out of the study.

Sensitive skin tests, by applying the product to healthy skin (arm region) of 50 volunteers for 48 hours, at least 10 of which were determined to have sensitive skin by the lactic acid stinging test. For each of the 5 compositions described in examples 1-5 above, no negative observed reaction was observed, and no volunteers dropped out of the study.

Described herein according to an embodiment is a composition for treatment of the skin, comprising at least two components of a superfood, a peptide, a hyaluronic acid (HA) or derivative thereof, and an agent selected from the group consisting of a prebiotic and a postbiotic. Optionally, the composition comprises both a prebiotic and a postbiotic. Optionally, the composition comprises: 2%-10% superfood; 0.00001%-5% peptide; 0.005%-4% HA; and 0.001%-3% prebiotics or postbiotics. Optionally, the superfood is selected from the group Punica granatum seed cell culture lysate, Morinda citrifolia callus culture lysate, Curcuma longa callus lysate, glycerin, citric acid, Olea vitae. Daucus carota sativa root cell culture lysate, Sarcocapnos crassifolia callus lysate, and Lithops pseudotruncatella callus lysate. Optionally, the peptide is selected from tetradecyl aminobutyroylvalylaminobutyric urea trifluoroacetate; palmitoyl tripeptide-5, palmitoyl tripeptide-5; palmitoyl dipeptide-5 diaminobutyroyl hydroxythreonine; butylene glycol, carbomer, coco-glucoside, glycerin, palmitoyl Tripeptide-7, palmitoyl Tetrapeptide-1; acetyl hexapeptide-8, Acetyl Hexapeptide-20, Copper Tripeptide-1, Acetyl Hexapeptide-30, Acetyl Hexapeptide-38, Acetyl Tetrapeptide-22, Acetyl Hexapeptide-37, Diaminopropionoyl Tripeptide-33, Acetyl Hexapeptide-51 Amide, Acetyl Tetrapeptide-2. Acetyl Heptapeptide-4, Acetyl Hexapeptide-49, Acetyl Tetrapeptide-40, Palmitoyl Hexapeptide-26, Acetyl Tetrapeptide-5, Palmitoyl Hexapeptide-19, Acetyl Cyclohexapeptide-34, Octapeptide-5, Palmitoyl Tripeptide-28, or a mixture thereof. Optionally, acetyl hexapeptide is present at 0.1%-1.015%, palmitoyl tripeptide-1 is present at 0.0001%-0.000203%, and palmitoyl tetrapeptide-7 is present at 0.00001%-0.0001015%. Optionally, the composition further comprises a probiotic. Optionally, the probiotic comprises lactobacillus. Optionally, the HA is selected from the group consisting of HA 100K-300K Da, HA 20K-50K Da, HA 5000K-20,000K Da, HA 800K-1500K Da, or a mixture thereof. Optionally, the HA is present in an amount of at least at 0.005%-4.0% of the composition. Optionally, the composition further comprises a sun protection factor (SPF) agent. Optionally, the skin treatment is a cosmetic treatment of wrinkles of the skin, dryness of the skin, dark spots of the skin, firmness of the skin, elasticity of the skin, and fading of the skin. Optionally, the treatment is applied as a cream, lotion, serum, cosmetic face mask, or cleanser.

Further described herein is a method for treatment of a skin condition comprising, applying to the skin at least two components selected from: a superfood, a peptide, a hyaluronic acid or derivative thereof, and an agent selected from the group consisting of a prebiotic and a postbiotic, thereby treating the skin condition. Optionally, the skin condition is selected from wrinkles, blemishes, roughness, dryness, fading, and dark circles firming. Optionally, the composition improves the condition of the skin selected from elasticity, skin thickness, and moisture. Optionally, the components are applied as a cream, lotion, serum, cosmetic face mask, or cleanser.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A composition for treatment of the skin, comprising at least two components selected from the group consisting of a superfood, a peptide, a hyaluronic acid (HA) or derivative thereof, and an agent selected from the group consisting of a prebiotic and a postbiotic.

2. The composition according to claim 1 comprising both a prebiotic and a postbiotic.

3. The composition of claim 1, comprising
2%-10% superfood;
0.00001%-5% peptide;
0.005%-4% HA; and
0.001%-3% prebiotics or postbiotics.

4. The composition of claim 1, wherein the superfood is selected from the group Punica granatum seed cell culture lysate, Morinda citrifolia callus culture lysate, Curcuma longa callus lysate, glycerin, citric acid, Olea vitae. Daucus carota sativa root cell culture lysate, Sarcocapnos crassifolia callus lysate, and lithops pseudotruncatella callus lysate.

5. The composition of claim 1, wherein the peptide is selected from tetradecyl aminobutyroylvalylaminobutyric urea trifluoroacetate; palmitoyl tripeptide-5, palmitoyl tripeptide-5; palmitoyl dipeptide-5 diaminobutyroyl hydroxythreonine; butylene glycol, carbomer, coco-glucoside, glycerin, palmitoyl Tripeptide-7, palmitoyl Tetrapeptide-1; acetyl hexapeptide-8, Acetyl Hexapeptide-20, Copper Tripeptide-1, Acetyl Hexapeptide-30, Acetyl Hexapeptide-38, Acetyl Tetrapeptide-22, Acetyl Hexapeptide-37, Diaminopropionoyl Tripeptide-33, Acetyl Hexapeptide-51 Amide, Acetyl Tetrapeptide-2, Acetyl Heptapeptide-4, Acetyl Hexapeptide-49, Acetyl Tetrapeptide-40, Palmitoyl Hexapeptide-26, Acetyl Tetrapeptide-5, Palmitoyl Hexapeptide-19, Acetyl Cyclohexapeptide-34, Octapeptide-5, Palmitoyl Tripeptide-28, or a mixture thereof.

6. The composition of claim 5, wherein acetyl hexapeptide is present at 0.1%-1.015%, palmitoyl tripeptide-1 is present at 0.0001%-0.000203%, and palmitoyl tetrapeptide-7 is present at 0.00001%-0.0001015%.

7. The composition of claim 1, further comprising a probiotic.

8. The composition according to claim 7, wherein the probiotic comprises lactobacillus.

9. The composition of claim 1, wherein the HA is selected from the group consisting of HA 100K-300K Da, HA 20K-50K Da, HA 5000K-20,000K Da, HA 800K-1500K Da, or a mixture thereof.

10. The composition of claim 1, wherein the HA is present in an amount of at least at 0.005%-4.0% of the composition.

11. The composition according to claim 1, further comprising a sun protection factor (SPF) agent.

12. A method for treatment of a skin condition comprising,
applying to the skin at least two components selected from: a superfood, a peptide, a hyaluronic acid or derivative thereof, and an agent selected from the group consisting of a prebiotic and a postbiotic,
thereby treating the skin condition.

13. The method of claim 12, wherein the skin condition is selected from wrinkles, blemishes, roughness, dryness, fading, and dark circles firming.

14. The method of claim 12, wherein composition improves the condition of the skin selected from elasticity, skin thickness, and moisture.

15. The method of claim 12, wherein the components are applied as a cream, lotion, serum, cosmetic face mask, or cleanser.

16. (canceled)

17. The composition according to claim 1, wherein the composition is a cream, lotion, serum, cosmetic face mask, or cleanser.
